(19) United States
(12) Patent Application Publication
(10) Pub. No.: US 2012/0101589 A1
(43) Pub. Date: Apr. 26, 2012

(54) PROSTHETIC DEVICES COATED WITH HEATED CROSS-LINKED FIBRIN

(75) Inventor: Raphael Gorodetsky, Jerusalem (IL)

(73) Assignee: HADASIT MEDICAL RESEARCH SERVICES AND DEVELOPMENT LTD., Jerusalem (IL)

(21) Appl. No.: 13/319,629

(22) PCT Filed: May 12, 2010
(86) PCT No.: PCT/IL2010/000379

§ 371 Date: Dec. 20, 2011

Related U.S. Application Data
(60) Provisional application No. 61/177,360, filed on May 12, 2009.

(51) Int. Cl. A61F 2/28; B05D 3/02

(52) U.S. Cl. 623/23.6; 427/2.24; 427/2.26

(57) ABSTRACT

The present invention relates to methods of coating prosthetic devices with dried fibrin. Particularly, the present invention relates to methods of coating the surface of prosthetic devices with fibrin and drying the fibrin-coated prosthetic devices at moderately-high temperatures for extended periods of time under low atmospheric pressure to obtain prosthetic devices coated with stable cross-linked fibrin capable of binding cells and thereby capable of integrating into tissues.

### FIELD OF THE INVENTION

The present invention relates to methods of coating prosthetic devices with dried fibrin. Particularly, the present invention relates to methods of coating the surface of prosthetic devices with fibrin and drying the fibrin-coated prosthetic devices at moderately-high temperatures for extended periods of time under low atmospheric pressure to obtain prosthetic devices coated with a stable cross-linked fibrin layer capable of binding cells and thereby capable of integrating into tissues.

### BACKGROUND OF THE INVENTION

Replacing or supplementing fractured, damaged, or degenerated mammalian skeletal bone with prosthetic implants made of biocompatible materials is commonplace in the medical arts. Most often, implant devices are intended to become permanently integrated into the skeletal structure. Unfortunately, permanent prosthetic attachment to bone is rare. Factors that influence long-term implant viability include material type used, bone fixation method, implant location, surgical skill, patient age, weight and medical condition. A plethora of devices have been constructed attempting to optimize these variables involved in producing an increase in bone fusion.

Common materials used in prosthetic devices include ceramics, polymers and metals. Currently, metallic materials afford the best mechanical properties and biocompatibility necessary for use as skeletal prosthetic implants. Frequently used metals include titanium and titanium alloy, stainless steel, gold, cobalt-chromium alloys, tungsten, tantalum as well as similar alloys. Titanium is popular in the implant field because of its superior corrosion resistance, biocompatibility, and because of its physical and mechanical properties compared to other metals. The dramatic increase over the last decade of titanium implants in neurosurgical, orthopedic and dental surgery attests to its acceptance as a prosthetic material. Titanium implants vary mostly in shape and surface type which influence the implant's ability to support load and to attach to bone.

A significant drawback of titanium implants is their tendency to loosen over time. There are three typical prevailing methods for securing metal prosthetic devices in the human body: press-fitting the device in bone, cementing them to an adjoining bone with methacrylate-type adhesives, or affixing in place with screws. All methods require a high degree of surgical skill. For example, a press-fitted implant must be placed into surgically prepared bone so that optimal metal to bone surface area is achieved. Patient bone geometry significantly influences the success of press-fitted implants and can limit their usefulness as well as longevity. Similar problems occur with cemented implants; furthermore, the cement itself is prone to stress fractures and is not bio-absorbable. Therefore, all methods are associated to varying degrees with cell lysis next to the implant surface with concomitant fibrotic tissue formation, prosthetic loosening, and ultimate failure of the device.

Currently, methods are being developed that produce osteointegration of bone to metal obviating the need for bone cements. Osteointegration is defined as bone growth directly adjacent to an implant without an intermediate fibrotic tissue layer. This type of biologic fixation avoids many complications associated with adhesives and theoretically would result in the strongest possible implant-to-bone bond. One common method is to roughen a metal surface creating a micro or macro-porous structure through which bone may attach or grow. Several implant device designs have been created attempting to produce a textured metal surface that allows direct bone attachment.

Metallic implant surfaces are also commonly coated with micro-porous ceramics such as hydroxyapatite (HA) or beta-tricalcium phosphate (TCP). The former treatment is more common because calcium-phosphate salts tend to be absorbed, in vitro, and thus loose their effectiveness. The HA coatings increase the mean interface strength of titanium implants as compared to uncoated implants. Despite the higher success rate of prosthetic devices coated with HA as compared to earlier implantation methods, failure over time still occurs. Again, proper integration requires that the surgeon create an exact implant fit into bone allowing the metal and bone surfaces to have maximum contact. Also, fibrotic tissue formation develops in some cases regardless of coating type.

Biopolymers such as fibrinogen and fibrin have been suggested as a coating material for metallic implants. For example, U.S. Pat. No. 5,324,647 to Rubens et al. disclose methods for coating surfaces of polymeric materials with fibrinogen wherein the coating is performed at a temperature of at least 56° C., but less than 100° C., in an atmospheric conditions to produce thermally denatured fibrinogen-coating. According to U.S. Pat. No. 5,324,647, the polymeric material can then be treated with thrombin to produce fibrin monomers. Optionally, the thrombin-treated surface can be exposed to a solution comprising factor XIII and additional fibrinogen, whereby the additional fibrinogen is converted to fibrin and cross-linked to the fibrin-coated surface. The resulting coated surface is presumed to be stable, anti-thrombotic and resistant to platelet adhesion.

U.S. Pat. No. 5,609,631 to Rubens et al. disclose methods for coating prosthetic surfaces with multimers of fibrin degradation products, preferably D-dimers. The methods according to U.S. Pat. No. 5,609,631 are useful for providing an anti-thrombogenic coating on prosthetic implants such as vascular grafts or artificial heart valves which are exposed to the circulating blood of a patient after implantation.

U.S. Pat. No. 5,660,873 to Nikolaychik et al. disclose methods for forming dried fibrin coating on a substrate such as a device for implantation in a body. According to U.S. Pat. No. 5,660,873, the substrate is contacted with thrombin and fibrinogen to form fibrin coating. The fibrin coating is then heated in ambient atmosphere to vaporize a substantial portion of the water whereby more than 80% of the fibrin is present in its native form.

Holmes et al. (J Am. Coll. Cardiol. 24: 525-531, 1994) disclosed fibrin-coated stents useful as a template for modifying the local response to arterial injury. The fibrin-coated stents according to Holmes et al. were prepared by dripping a fibrinogen solution and a thrombin solution on a tantalum stent and after fibrin polymerized, the fibrin was soaked in a heparin solution.

Marx et al. (J Biomed. Mater. Res. 84B: 49-57, 2008) disclosed the conformational changes associated with moderate heating (47° C.-60° C.) of fibrinogen bound to plastic ELISA plates. The results indicated that heat denaturation of fibrinogen bound to plastic exposed a C-terminal epitope (γ₃₉₇₋₄₁₁) as well as Haptides epitopes (β₄₆₃₋₄₈₃ and γ₃₇₂₋₃₉₁) which helped to attract cells.

There is still a need for improved methods for coating fibrin on prosthetic devices which result in stable fibrin coating.

### SUMMARY OF THE INVENTION

The present invention provides improved methods for coating fibrin on the surface of prosthetic devices, which devices are useful in orthopedic or dental surgery.

The present invention is based in part on the observation that coating of metal prosthetic devices with fibrinogen which contains factor XIII and then contacting the fibrinogen-coated metal prosthetic devices with thrombin resulted in the formation of a layer of a fibrin gel which upon drying under vacuum at temperatures between 70° C. to 80° C. for extended periods of time of 8 to 16 hours yielded an advantageous heat-stabilized cross-linked fibrin coating. This coating is a dehydrated thermally stabilized fibrin designated herein as dehydrothermal fibrin. The heat-stabilized fibrin coating was more resistant to protein degradation than a fibrin coating which was not subjected to drying conditions under vacuum. Moreover, the heat-stabilized fibrin coating was highly efficacious in supporting cell attachment and cell proliferation on the coated prosthetic devices. Without wishing to be bound by any theory of mechanism of action it is postulated that the vacuum drying may be advantageous due to improvement in the porosity of the coating thereby allowing better attachment or proliferation of the cells.

The present invention further discloses that metal prosthetic devices coated with the heat-stabilized or dehydrothermal cross-linked fibrin can be sterilized with no detectable change in the cell attachment efficacy. Rehydration of the dehydrothermal cross-linked fibrin coating by immersing the coated devices in an aqueous solution had an insignificant effect on the fibrin coating stability as compared to prosthetic devices which were coated with fibrin under heating at the same temperatures but at atmospheric conditions.

The present invention further discloses that dehydrothermal cross-linked fibrin coating on titanium prosthetic devices covered with titanium oxide improved significantly cell attachment to these prosthetic devices. While titanium prosthetic devices covered with titanium oxide are commonly used in orthopedic and dental surgery by virtue of their improved efficacy to attract cells, the present invention discloses that the heat-stabilized or dehydrothermal cross-linked fibrin coating on titanium screws covered with titanium oxide enhanced cell attachment even further. Similarly, coating of CaSO4 granules with dehydrothermal cross-linked fibrin resulted in a significantly higher attachment of mesenchymal stem cells to the fibrin coated granules than to uncoated granules. Thus, coating of prosthetic devices with dehydrothermal cross-linked fibrin is highly advantageous for osteointegration.

According to one aspect, the present invention provides a method for coating a surface of a prosthetic device with dehydrothermal cross-linked fibrin comprising the steps of:
(i) contacting a prosthetic device with a first aqueous solution comprising fibrinogen and factor XIII;
(ii) contacting the prosthetic device of step (i) with a second aqueous solution comprising thrombin;
(iii) drying said prosthetic device of step (ii) at a temperature ranging from about 60° C. to about 90° C. under pressure lower than atmospheric pressure for at least 4 hours, thereby yielding dehydrothermal cross-linked fibrin.

According to some embodiments, fibrinogen is present in the first aqueous solution at a concentration ranging from about 2 mg/ml to about 75 mg/ml. According to additional embodiments, the fibrinogen is present in the first aqueous solution at a concentration ranging from about 5 mg/ml to about 20 mg/ml.

According to further embodiments, the thrombin is present in the second aqueous solution at a concentration ranging from about 0.001 IU/ml to about 200 IU/ml. According to yet further embodiments, thrombin is present in the second aqueous solution at a concentration ranging from about 1 IU/ml to about 100 IU/ml, alternatively at a concentration ranging from about 10 IU/ml to about 50 IU/ml.

According to yet further embodiments, the drying is performed at a temperature ranging from about 65° C. to about 85° C. According to an exemplary embodiment, the drying is performed at a temperature ranging from about 70° C. to about 80° C. According to a certain embodiment, the drying is performed under vacuum.

According to still further embodiments, the drying is performed for a duration ranging from about 4 hours to about 24 hours, alternatively from about 6 to 20 hours, further alternatively from about 8 hours to about 16 hours.

According to another embodiment, contacting the prosthetic device with the first and/or second aqueous solutions is performed by immersing the device in said solutions. According to a further embodiment, contacting the prosthetic device with the first and/or second aqueous solutions is performed by spraying the solutions on the device. According to a certain embodiment, contacting the prosthetic device with the first aqueous solution is performed by immersing and with the second aqueous solution by spraying.

According to a further embodiment, the first and/or second aqueous solution further comprise a calcium salt. According to still further embodiments, the aqueous solution further comprises at least one additive and/or a pharmacological agent. Among the pharmacological agents that can be used, agents that stimulate bone, cartilage and/or endothelial cell growth, anti-inflammatory agents, blood clotting inhibitors, antibiotic agents, and antineoplastic agents are preferred.

According to further embodiments, the first aqueous solution further comprises a moderate detergent, optionally further comprising sodium chloride.

According to one exemplary embodiment, the first aqueous solution comprises fibrinogen, factor XIII, and a calcium salt and the second aqueous solution comprises thrombin. According to another exemplary embodiment, the first aqueous solution comprises fibrinogen and factor XIII, and the second aqueous solution comprises thrombin and a calcium salt. According to a further exemplary embodiment, the first aqueous solution comprises fibrinogen, factor XIII, polysorbate 80 at a concentration of about 1% to about 5%, preferably at a concentration of about 2%, CaCl₂ at a concentration of about 1 mM to about 30 mM, preferably at a concentration of about 2 mM, NaCl at a concentration of about 0.2 M to 0.5 M, preferably at a concentration of 0.3 M, and Tris buffer.

According to another embodiment, the method can further comprise the step of rehydrating the dried fibrin in an aqueous solution. It is to be appreciated that the aqueous solution for rehydrating the fibrin coating can comprise at least one additive and/or a pharmacological agent.

According to a certain embodiment, the prosthetic device is an artificial bone implant, preferably comprises metallic material, more preferably titanium. Alternatively, the prosthetic device is a prosthetic matrix including, but not limited to, CaSO₄.

According to another aspect, the present invention provides a prosthetic device coated with dehydrothermal cross-linked fibrin prepared according to the methods of the present invention.

According to another aspect, the present invention provides a method for treating a tissue defect or lesion in a mammalian subject comprising implanting into the tissue defect or lesion a prosthetic device coated with dehydrothermal cross-linked fibrin prepared according to the principles of the present invention.

According to some embodiments, the tissue defect or lesion is a bone lesion. According to a certain embodiment, the bone lesion is within a tooth. According to further embodiments, the tissue defect or lesion is a cartilage lesion.

According to yet further embodiment, the mammalian subject is a human. According to still further embodiment, the mammalian subject is an animal.

According to further aspect, the present invention provides a prosthetic device coated with dehydrothermal cross-linked fibrin for treating a tissue defect or lesion in a mammalian subject, the prosthetic device coated with dehydrothermal cross-linked fibrin prepared by the methods of the present invention.

These and other embodiments of the present invention will be better understood in relation to the figures, description, examples and claims that follow.

### BRIEF DESCRIPTION OF THE FIGURES

FIGS. 1A-K show light, SEM and fluorescence photomicrographs of the surface of titanium screws coated with different concentrations of fibrin to which foreskin fibroblasts were attached. To detect cell attachment, the fibroblasts were stained with propidium iodide. FIGS. 1A-B show light and fluorescence photomicrographs, respectively, of control screws. FIGS. 1C-D show light and fluorescence photomicrographs, respectively, of screws coated with 10 mg fibrin. FIGS. 1E-F show light and fluorescence photomicrographs, respectively, of screws coated with 20 mg fibrin. FIG. 1G shows light photomicrograph of untreated surface. FIGS. 1I and 1K show SEM of fibrin coated screws showing cell attachment.

FIG. 2 shows fibroblast attachment to uncoated or fibrin-coated titanium screws. Brushed titanium screws were coated with different concentrations of fibrin and the number of foreskin fibroblasts attached to the screws was measured by a modified MTS colorimetric assay for cell number.

FIGS. 3A-C show fibroblast attachment and proliferation on control or fibrin-coated titanium screws covered with titanium oxide. Titanium screws covered with titanium oxide were coated with fibrin and the number of fibroblasts attached to these screws or to uncoated screws one day or 3 days after cell addition was measured by a modified MTS assay (FIG. 3A). Cell nuclei of the attached cells to control screws (FIG. 3B) or to fibrin-coated screws (FIG. 3C) were stained with PI and florescence photomicrographs are shown.

FIG. 4 shows mesenchymal stem cell attachment to fibrin coated CaSO₄ granules. CaSO₄ granules were coated with different concentrations of fibrin and mouse mesenchymal stem cells were added for 24 hours. Cell attachment was measured by MTS assay.

### DETAILED DESCRIPTION OF THE INVENTION

The present invention provides methods for coating fibrin on the surface of prosthetic devices so as to obtain fibrin-coated prosthetic devices useful for creating connections to tissue parts, in particular to bone parts, cartilage parts, tendon parts, ligament parts, but also to parts of other tissues, wherein the prosthetic devices are able to provide stable connections after implantation.

According to one aspect, the present invention provides a method for coating a surface of a prosthetic device with dried fibrin, the method comprising:
(a) contacting a prosthetic device with a first aqueous solution comprising fibrinogen and factor XIII;
(b) contacting the prosthetic device coated with fibrinogen of step (a) with a second aqueous solution comprising thrombin; and
(c) drying the prosthetic device of step (b) coated with fibrin at a temperature ranging from about 60° C. to about 90° C. for extended periods of time of at least about 4 hours at pressure lower than atmospheric pressure, thereby yielding a prosthetic device coated with heat stabilized, dehydrothermal cross-linked fibrin which is substantially devoid of water.

The term “dehydrothermal” fibrin coating refers to a fibrin layer prepared by heating the fibrin-coated devices at pressure lower than atmospheric pressure, typically under partial or full vacuum, so as to produce a dried, hardened and stabilized fibrin coating substantially devoid of water.

The term “substantially devoid” of water refers to the dried fibrin coating having up to about 2% (w/w) water. Preferably, the dried or dehydrothermal fibrin coating has up to about 1% of water (w/w), more preferably of up to about 0.1% of water (w/w).

The term “about” refers to ±10% of the indicated value.

It is to be understood that while the fibrin coating according to the principles of the present invention was formed by heating the fibrin coated prosthetic devices under vacuum so as to yield heat stabilized fibrin coating, fibrin coating disclosed in previous studies was formed by heating the fibrinogen coated devices at atmospheric conditions and only then adding thrombin. Without wishing to be bound to any mechanism of action, it is postulated that fibrin coating according to the present invention enables the formation of a porous fibrin layer capable of attracting high number of cells. It is further to be understood that the degree of fibrin cross-linking may be optimized during the coating procedures disclosed herein by varying the concentrations of fibrinogen and/or factor XIII and/or thrombin, and thereby affecting cell attachment.

The prosthetic material useful for practicing the present invention can be any material that is safe for use in a living body. The prosthetic material can be a metal, such as stainless steel, titanium, alloys of steel, nickel, titanium, molybdenum, cobalt and chromium, and nitinol (nickel-titanium alloy). Alternatively, the prosthetic material can be a polymeric material such as polyethylene terephthlate, polyethylene, polyurethane, polylactic acid, polyglycolic acid, or polytetrafluoroethylene. The prosthetic material can also be a biodegradable inorganic material such as calcium sulfate (hemi- or di-hydrate form), salts of calcium phosphate such as tricalcium phosphate or hydroxyapatite, and blends or combinations thereof.

The prosthetic devices to be coated with fibrin include, but are not limited to, screws, pins, stents, bone implants, artificial vascular implants, duct implants, urological implants, internal organs, heart valves, or other artificial prosthetic structures, including those that are exposed to blood flow after implantation. Artificial duct implants that can be coated according to the methods of the invention include artificial urinary ducts, artificial kidney tubules, artificial lymphatic ducts, artificial bile ducts, artificial pancreatic ducts, indwelling catheters, shunts and drains.

To form the fibrin coating on the prosthetic device, the prosthetic device is contacted with a first solution which comprises fibrinogen and factor XIII, the first solution is substantially devoid of thrombin, and then contacted with a second solution which comprises thrombin, the second solution is substantially devoid of fibrinogen and factor XIII.

The terms “substantially devoid” or “substantially free” of fibrinogen or thrombin refer to a solution having fibrinogen or thrombin at amounts of up to about 0.2 mg/ml or up to about 0.0001 U/ml, respectively. It has been discovered that contacting the device with fibrinogen and factor XIII before thrombin yields a fibrin coating that strongly adheres to the prosthetic device surface as compared to a fibrin coating formed by contacting the device simultaneously with fibrinogen, factor XIII and thrombin.

The first and second solutions are preferably aqueous solutions having a pH ranging from about 6.8 to about 7.8. It is to be understood that while the first solution can comprise both fibrinogen and Factor XIII, the present invention also encompasses two different solutions, one comprising fibrinogen and the another comprising Factor XIII. Fibrinogen can be isolated from plasma by known procedures such as the Cohn fractionation procedure (i.e., 8-10% ethanol at 4° C. added to non-coagulated plasma). Alternatively, an aqueous solution of fibrinogen containing endogenous Factor XIII can be obtained from commercial sources such as Baxter/Immuno, Behringwerke, Omrix or the like. Preferably, the fibrinogen is human fibrinogen, more preferably the human fibrinogen is autologous, i.e., derived from the patient. According to a certain embodiment, the solution comprising autologous fibrinogen further comprises autologous factor XIII. Alternatively, fibrinogen can be recombinant fibrinogen prepared by methods known in the art.

The concentration of the fibrinogen ranges from about 2 to about 75 mg/ml, preferably from about 3 to about 50 mg/ml, more preferably from about 4 to about 30 mg/ml, and most preferably from about 5 to about 20 mg/ml. The solution of fibrinogen is preferably substantially free of thrombin, preferably having a concentration of thrombin that is not more than about 0.0001 IU/ml.

Factor XIII can be prepared from plasma according to known methods, such as those disclosed by Cooke and Holbrook (Biochem. J. 141: 79-84, 1974) and Curtis and Lorand (Methods Enzymol. 45: 177-191, 1976), incorporated by reference as if fully set forth herein. The a₂ dimer form of factor XIII can be prepared from placenta as disclosed in U.S. Pat. Nos. 3,904,751; 3,931,399; 4,597,899 and 4,285,933, incorporated by reference as if fully set forth herein. Alternatively, recombinant factor XIII can be used. Preparation of recombinant factor XIII is known in the art, see, for example, Davie et al., EP 268,772 incorporated by reference as if fully set forth herein. It is to be understood that any enzyme or protein that can cross-link proteins by tranglutamination, including, but not limited to tissue transglutaminase, is encompassed in the present invention. Preferably, Factor XIII is present at a concentration ranging from about 5 to about 500 IU/ml.

The concentration of the thrombin ranges from about 0.001 to about 200 IU/ml, preferably from about 0.05 IU/ml to about 150 IU/ml, more preferably from about 1 IU/ml to about 100 IU/ml, and most preferably from about 10 IU/ml to 50 IU/ml. The solution of thrombin is substantially free of fibrinogen, preferably having a concentration of fibrinogen that is not more than about 0.2 mg/ml. As an alternative of thrombin, equivalent proteases such as snake venom proteases (e.g., reptilase) can be used.

The first and second solutions can further comprise a salt to stabilize the fibrinogen and/or thrombin in the solutions. The stabilizing salt can be any salt including, but not limited to, calcium chloride, sodium chloride, magnesium sulfate, sodium sulfate, potassium chloride, (hydroxymethyl) aminomethane (Tris), and mixtures thereof Preferably, the salt is calcium chloride ranging from about 1 to about 30 mM. The first and/or second aqueous solutions can further comprise NaCl at a concentration of about 0.2 to about 0.5 M, preferably at a concentraiotn of 0.3 M. According to a certain embodiment, the second solution comprises thrombin and calcium chloride. It is to be appreciated that as a consequence of the interaction of thrombin with fibrinogen in the presence of factor XIII and calcium ions, fibrinogen is converted to fibrin monomers which are then cross-linked to form the fibrin matrix. In addition, it has been discovered that contacting the device with fibrinogen and factor XIII before the addition of thrombin and Ca²⁺ yields a fibrin coating that strongly adheres to the prosthetic device surface as compared to a fibrin coating formed by contacting the device simultaneously with fibrinogen, factor XIII, thrombin and Ca²⁺.

As will be appreciated, the first solution comprising fibrinogen can further comprise one or more additives such as enzyme inhibitors (e.g., aprotinin, ε, aminocaproic acid), buffering agents (e.g., phosphate, acetate, Tris or citrate), anti-oxidants (e.g., ascorbic acid or sodium metabisulfite), preservatives (e.g., Thimerosal, benzyl alcohol, parabens, m-cresol), detergents (e.g., Tween 80; final concentration of about 1% to about 5%), and/or one or more pharmaceutical agents including, but not limited to, agents that stimulate bone, cartilage and/or endothelial cell growth, anti-inflammatory agents, blood clotting inhibitors, antibiotic agents, and antineoplastic agents, depending upon the desired properties of the coating or the desired effect of the coating on the patient.

To form the fibrin coating on the surface of a prosthetic device, the prosthetic device can be immersed sequentially in the first and second solutions. Immersing the prosthetic device in the first solution forms a liquid coating of fibrinogen on the device. The fibrinogen coating forms fibrin when the device is contacted with the second solution comprising thrombin.

During immersion of the device in the first solution, the temperature of the solution preferably ranges from about 21° C. to about 37° C., and the time of immersion preferably ranges for about 10 minutes to about 1 hour or more.

During immersion of the device in the second solution, the temperature of the solution is preferably maintained from about 21° C. to about 37° C., and the time of immersion preferably ranges from about 10 minutes to 1 hour or more.

There are other methods to contact the device with fibrinogen and thrombin. Fibrinogen and thrombin can also be contacted with the device by spraying the first and/or second solutions on the device. As will be appreciated, the fibrinogen solution and the thrombin solution can also be contacted with the device by a combination of immersion and spraying. Preferably the device is immersed in the first solution comprising fibrinogen and then the second solution comprising thrombin is sprayed, thereby generating a stable fibrin coating.

After formation of the fibrin coating, the coating is dried at a temperature of about 60° C. to 90° C., preferably at about 70° C. to about 80° C., and at pressure lower than atmospheric pressure, preferably, under vacuum e.g., about 28 mm Hg vacuum.

It should be appreciated that removal of water from the fibrin coating has several advantages, including the ability to store the fibrin coating for extended periods of time before use and the increased adhesion of the coating to the device surface.

Removal of water and heating is also believed to increase fibrin cross-linking due to thermal cross-linking. The term “thermal cross-linked” or “dehydrothermal cross-linked” protein refers to a protein having new bonds formed upon dehydration and heating of the protein. Examples of new bonds that can be formed upon dehydration and heating of a protein include amide bonds between amino and carboxyl groups or covalent bonds between hydroxyl groups. It is to be understood that while factor XIII is known to induce cross-linking of fibrin monomers to from the fibrin polymer, i.e., fibrin matrix, the present invention provides a method for increasing the bonding of the fibrin polymer by thermal cross-linking.

The time of drying is selected so as to form fibrin coating which is substantially devoid of water. The term “substantially devoid” of water refers to a fibrin coating which comprises at most residual amounts of water compared to the amount present in the fibrin coating gel before drying. The term “residual amount” as used herein is meant to indicate that water constitutes, not more than 2% w/w of fibrin coating, preferably, water constitutes not more that 1% w/w of the fibrin coating, and more preferably not more than 0.1% w/w of the fibrin coating. Thus, the time of drying is selected such that drying reduces the water content of the coating to at least 2% w/w, preferably to at least 1% w/w, and more preferably to at least 0.1% w/w of the fibrin coating. The time of drying ranges from about 4 hours to about 24 hours, alternatively from about 6 hours to 20 hours, or from about 8 hours to about 16 hours. Alternatively, the time is also selected such that at least about 30% by weight of the fibrin is denatured after drying, further alternatively at least about 40%, 50%, 60% or at least 70% by weight of the fibrin in the fibrin coating is denatured after drying.

The prosthetic device coated with the dried heated fibrin can optionally be sterilized by methods known in the art, for example, by immersing the device in ethanol, preferably for at least 30 minutes. Alternatively, the device can be sterilized by gamma irradiation, preferably with at least about 0.5×10⁶ cGy. According to a certain embodiment, the gamma irradiation is up to 2×10⁶ cGy.

Before implantation of the fibrin coated prosthetic device in a body, the prosthetic device can be immersed in an aqueous solution such as water, buffer or a culture medium, optionally comprising one or more additives listed herein above and/or one or more pharmacological agents.

Following the replenishment of water to the coating, the fibrin coating can be seeded with cells of mesodermal origin, such as stem cells, osteoblasts, chondrocytes and/or endothelial cells to improve the implantation of the prosthetic device. Osteoblasts, chondrocytes and endothelial cells can be obtained by standard procedures known in the art. For seeding, the cells can be cultured on the fibrin-coated device, the latter can be incubated in culture medium, generally at a temperature of about 37° C. in an atmosphere containing about 5% to 10% carbon dioxide. Satisfactory attachment of the cells to the fibrin coated device can be obtained within about 4 to about 24 hrs. After seeding, the fibrin coated device can be further incubated to allow the cells to replicate.

The pharmacological agents encompassed in the present invention include, but are not limited to, agents that stimulate bone, cartilage and/or endothelial cell growth, anti-inflammatory agents, blood clotting inhibitors, antibiotic agents, and antineoplastic agents.

Agents that stimulate osteoblast or chondrocyte growth include, but are not limited to, TGFβ, platelet derived growth factor, and bone morphogenic protein.

Agents that stimulate endothelial cell growth include a variety of extracellular matrix proteins as well as chemotactic and/or cell growth factors. Specific proteins contemplated for use in this manner include, but are not limited to, basic fibroblast growth factor, endothelial cell growth factor, α2 macroglobulin, vitronectin, fibronectin, cell-binding fragments of fibronectin, and derivatives and mixtures thereof.

Anti-inflammatory agents that suppress inflammation of tissue after implantation of the prosthetic device include antihistamines, glucocorticoids, non-steroid anti-inflammatory agents, salicylates, steroids, and derivatives and mixtures thereof. The anti-inflammatory agent can be used at pharmacological concentrations.

Blood clotting inhibitors (e.g., anti-coagulants) that inhibit the formation of blood clots after implantation of the prosthetic device include heparin and its fractions, recombinant hirudin, hirulog-1, D-phenylalanyl-L-prolyl-L-arginyl chloromethyl ketone, dipyridamole, RGD-like peptide, and derivatives and mixtures thereof. The blood clotting inhibitors are generally used at pharmacological concentrations to prevent clotting. The blood clotting inhibitor heparin preferably has a concentration ranging from about 10 to about 500 IU/ml, preferably about 50 to about 250 IU/ml, and more preferably from about 75 to about 150 IU/ml. Dipyridamole preferably has a concentration ranging from about 10 to about 100 moles/ml.

Antibiotic agents are used to prevent infection after implantation of the prosthetic device. Preferred antibiotics include all broad and medium spectrum agents, including aminoglycolides, cephalosporons (1st, 2nd, and 3rd generation), macrolides, penicillins, tetracyclines, and derivatives and mixtures thereof. The antibiotic is generally used at pharmacological concentrations.

The present invention encompasses contacting the fibrin-coated prosthetic device of step (b) with a cross-linking agent. According to some embodiments, the additional cross-linking reaction involves carbohydrate groups and free amino groups of fibrin(ogen). This cross-linking can be performed by immersing the fibrin-coated device in a solution containing a cross-linking agent including, but not limited to, potassium periodate, 1-ethyl-(3,3-dimethylaminopropyl)carbodiimide (EDC), chloro-1-methyl-pyridinium iodide (CPMI; see, for example, Young et al. J. Biomaterials Sci. Polymer Edn. 15: 767-780, 2004) or CNBr (a reagent which is commonly used to couple proteins to Sepharose).

### EXAMPLE 1

### Coating with Fibrin

Fibrinogen was isolated from human plasma by cold precipitation with ˜8-10% ethanol. To increase the Fibrinogen content to >80% of clottable proteins, a second precipitation of the recovered proteins with ˜8-10% ethanol was performed. The fibrinogen thus isolated contained endogenous levels of factor XIII that acts as a cross-linking enzyme after the formation of fibrin monomers.

Fibrin coating was performed in either one of the following methods:
a. The implant to be coated was immersed in a fibrinogen solution (5-20 mg/ml) to enable adsorption of fibrinogen to the surface of the implant. Thereafter, thrombin (final concentration 5-10 units/ml) was added and mixed. The implant was removed immediately, before clotting occurred, enabling a thin film of fibrin to be formed on the surface.
b. The implant to be coated was immersed in a fibrinogen solution (5-20 mg/ml) to enable adsorption of fibrinogen to the surface of the implant. Thereafter, the implant was removed from the fibrinogen solution and extra liquid was allowed to drain. The implant was sprayed shortly with a thrombin solution of 40 units/ml.

The object that was coated with a thin layer of fibrin gel was allowed to dry and positioned in a vacuum oven at 70-80° C. for about 8-16 hours.

In some cases, methods (a) or (b) were repeated so as to obtain another layer of fibrin coating on the implant. Thereafter, the implant was positioned again in the vacuum oven under the same conditions as described herein above.

After the drying step, the implant was sterilized by either one of the following methods:
a immersion in ethanol for at least 3 hrs;
b. gamma irradiation with 0.5-2×10⁶ cGy (5-20 KGy).

Before application, the coated implant was equilibrated in an aqueous medium causing limited hydration of the fibrin without compromising the stable coating.

### EXAMPPLE 2

### Cell Binding to Fibrin-Coated Titanium Screws

Metal (titanium) screws for teeth implants have to be integrated in the living bone in an optimal manner so as to obtain a high binding force to the bone tissue. Such effect could be achieved by increased attraction of cells to the implant. The commercially available threaded screws are made of titanium covered with roughened surface of titanium-oxide to increase their surface area. Such screws have relatively good cell binding properties. Nevertheless, increasing their cell binding efficiency can contribute to their better and faster integration in the bones.

The aim of this study was to determine whether coating titanium screws with stable and durable fibrin film can improve cell binding to the fibrin-coated screws and hence enhance incorporation of the screw into the target injured tissue.

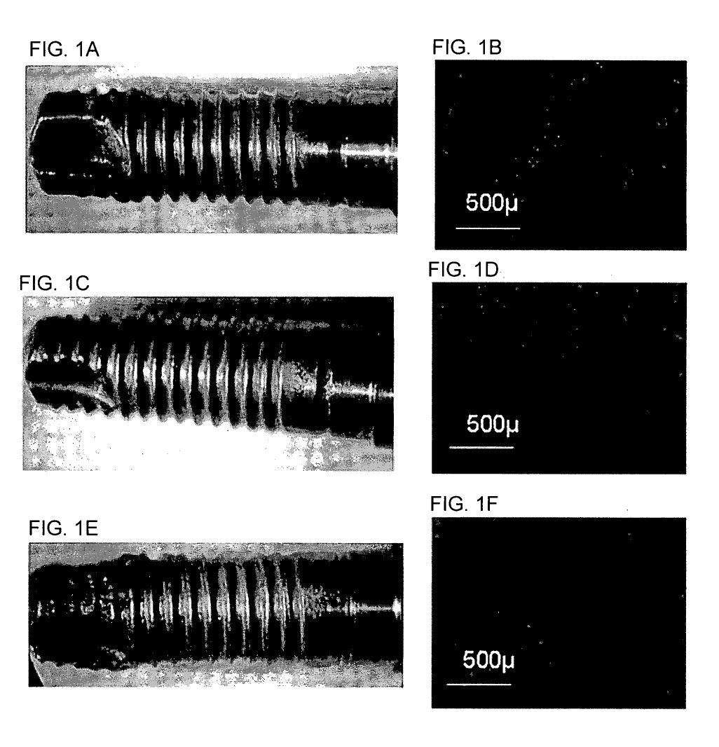

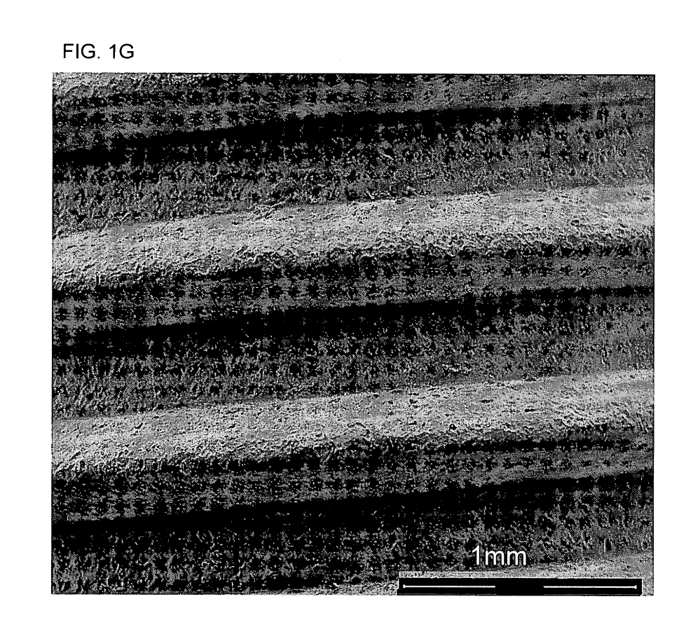

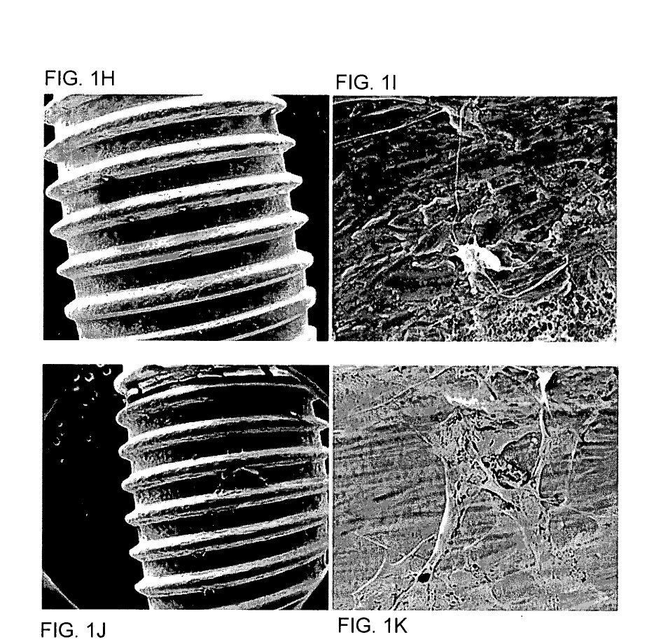

For this end, commercially available titanium (Ti)-screws were treated to expose their engraved Ti surface. The screws were brushed with metal brushes and cleaned extensively and further exposed to a concentrated acid (HNO₃). As a result, the Ti surface of the screws was exposed (FIG. 1).

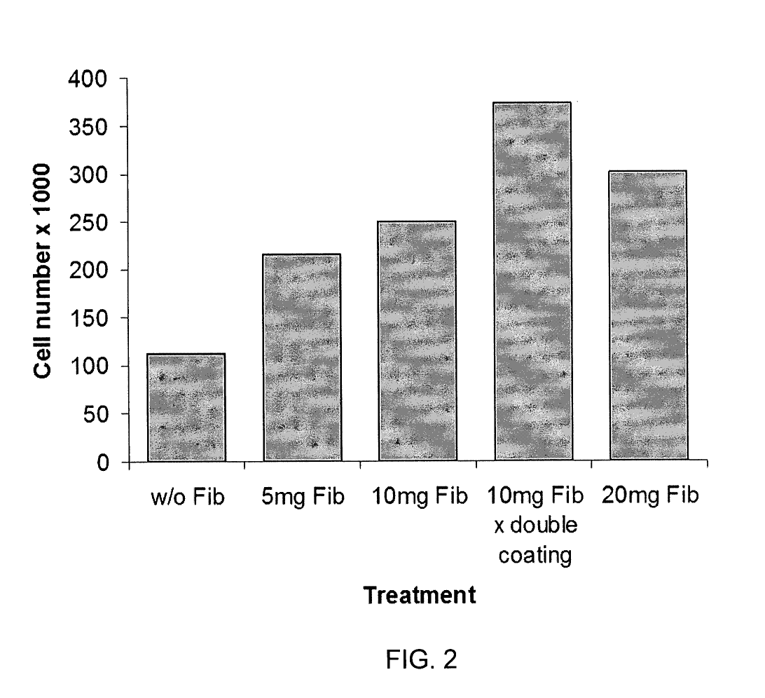

The screws were then coated with fibrin at different concentrations of fibrinogen (mg/ml): 5, 10, 20, and 2 cycles of coating with 10 mg/ml fibrinogen. After coating, drying and sterilizing, the screws were incubated with cultured foreskin fibroblasts (FF) as a model for mesenchymal cells for 24 hrs and the non-attached cells were removed. Typically, the cellularized implant was allowed to stabilize for additional 12-24 hrs. Cell number was then determined by a modified MTS assay (Gorodetsky et al., Methods Mol. Biol. 238: 11-24, 2004) which evaluates cell number on matrices. The screws were also viewed by scanning electron microscopy (SEM; FIG. 1), and the nuclei of the cells were stained by propidium iodide (PI) to visualize cell density (FIG. 2).

As shown in FIG. 2, coating of titanium screws with 20 mg fibrin increased cell binding to the screws by ˜3 fold.

### EXAMPLE 3

### Cell Binding and Proliferation on Intact Titanium Screws Coated with Fibrin

The aim of this study was to determine whether fibrin coating of commercially available titanium screws covered with titanium oxide can improve cell binding and enable cell proliferation. Cell binding efficiency to these screws is expected to be higher due to the oxidized-Ti coating and especially due to the fine engraved surfaces.

Six screws were used as a control and six screws were coated twice each with 10 mg/ml fibrinogen. The screws were tested one day after cell loading (3 screws) and on day 3 to check proliferation (3 screws).

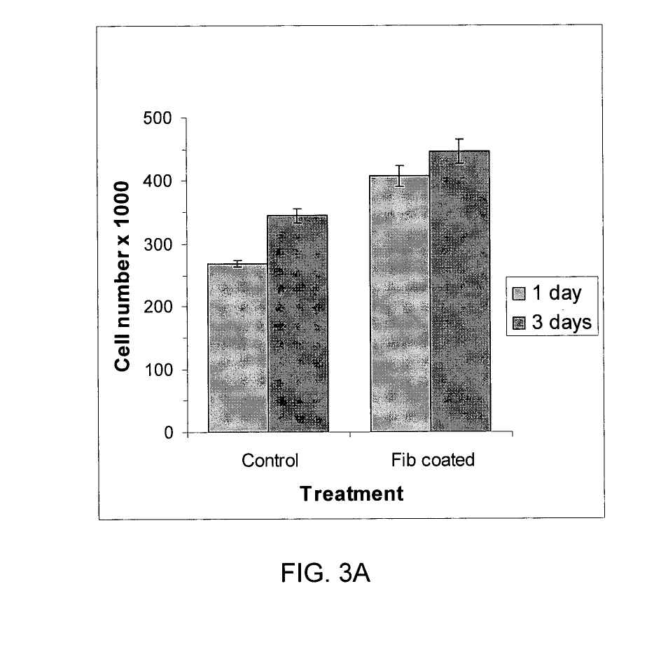

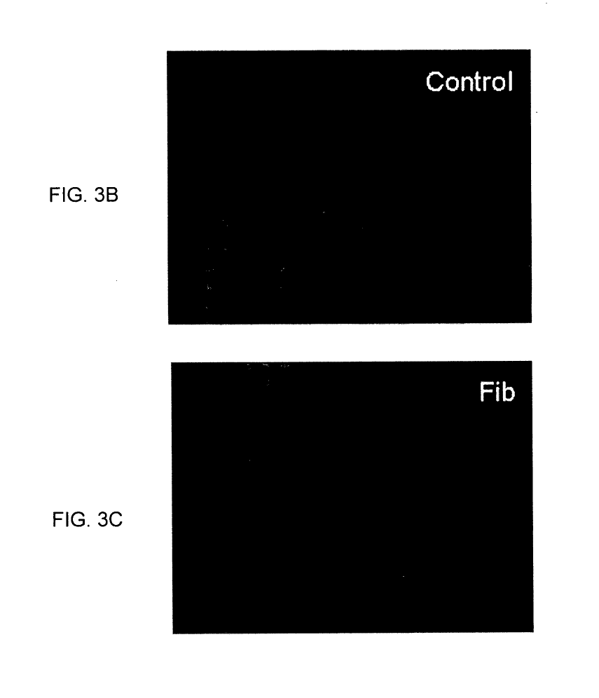

As shown in FIG. 3, the number of cells that adhered titanium screws covered with titanium oxide was higher than that attached to the brushed screws (compare to FIG. 2) confirming that the oxidized Ti coating improves cell attachment. FIG. 3 also shows that cell attachment to the heat treated fibrin-coated screws was significantly higher than the number of cells adhered to the intact screws (FIG. 3, 1 day). Moreover, the cells were shown to be able to proliferate on the coated screws (FIG. 3, 3 days). Thus, fibrin coating on titanium screws covered with titanium oxide improves significantly the ability of cells to attach and proliferate on these screws.

### EXAMPLE 4

### Coating of Granular CaSO₄ with Fibrin by the Dehydrothermal Reaction for Bone Regeneration

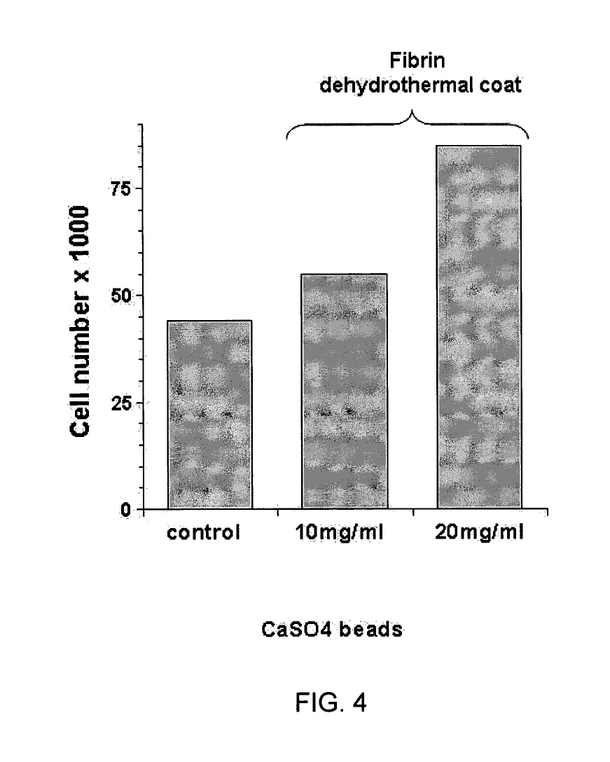

CaSO₄ granules for bone regeneration (Class Implant, Rome, Italy) were immersed in a solution containing 10 or 20 mg/ml fibrinogen for 1 hr. The residual fibrinogen was discarded and thrombin (40 Units/ml) was added. The granules were incubated in a vacuum oven at 85° C. for 6 to 8 hrs. Thereafter, the granules were recovered and disaggregated by mild mechanical force. Samples (10 mg each) of the treated granules were sterilized overnight in ethanol and rehydrated in medium containing 20% FCS. The granules were then exposed to mouse mesenchymal stem cells (mMSC; 200,000 cells) for 24 hrs. Non-attached cells were discarded and the granules were assayed for cell number by the MTS assay. As indicated in FIG. 4, fibrin coating increased cell binding to the granules.

### EXAMPLE 5

### Coating of Titanium Screws with Fibrin by Heating under Atmospheric Conditions vs. Vacuum

Titanium screws covered with titanium oxide are immersed in a solution containing 1, 5, 10 or 20 mg/ml fibrinogen in Tris buffer for 1 hr. The residual fibrinogen is discarded and thrombin (40 Units/ml) and 2 mM Ca²⁺ are added. The screws coated with 1 or 5 mg/ml fibrinogen are incubated in an oven at 85° C. under atmospheric conditions for 5 to 10 minutes and the screws coated with 10 or 20 mg/ml fibrinogen are incubated in a vacuum oven at 85° C. for 6 to 18 hrs. Thereafter, the screws are incubated in acetate buffer at different pHs, e.g., 3 to 7 for 30 minutes. In addition, the screws are incubated in Tris buffer, pH 7.0 in the presence of various proteolytic enzymes. Thereafter, the amount of fibrin degradation products released to the buffer is measured. Coating of titanium screws with fibrin under heating and vacuum conditions enables the formation of a more stable and less degradable fibrin layer as compared to the fibrin layer formed under heating at atmospheric conditions. Thus, coating of fibrin under heating and vacuum condition provides stable and durable fibrin coating.

It will be appreciated by persons skilled in the art that the present invention is not limited by what has been particularly shown and described herein above. Rather the scope of the invention is defined by the claims that follow.

1. A method of coating a surface of a prosthetic device with dehydrothermal cross-linked fibrin comprising the steps of:
(i) contacting a prosthetic device with a first aqueous solution comprising fibrinogen and factor XIII;
(ii) contacting the prosthetic device of step (i) with a second aqueous solution comprising thrombin; and
(iii) drying said prosthetic device of step (ii) at a temperature ranging from about 60° C. to about 90° C. for at least 4 hours under pressure lower than atmospheric pressure, thereby yielding dehydrothermal cross-linked fibrin.

2. The method of claim 1, wherein the fibrinogen is present in the first aqueous solution at a concentration ranging from about 2 mg/ml to about 75 mg/ml.

3. (canceled)

4. The method of claim 1, wherein the thrombin is present in the second aqueous solution at a concentration ranging from about 0.001 IU/ml to about 200 IU/ml.

5-6. (canceled)

7. The method of claim 1, wherein the drying is performed under vacuum.

8-9. (canceled)

10. The method of claim 1, wherein contacting the prosthetic device with the first solution is performed by immersion.

11. The method of claim 1, wherein contacting the prosthetic device with the second solution is performed by spraying.

12. The method of claim 1, wherein the first aqueous solution further comprises a calcium salt.

13. The method of claim 1, wherein the first aqueous solution further comprises at least one additive and/or a pharmacological agent.

14. The method of claim 1, wherein the second aqueous solution comprises thrombin and a calcium salt.

15. The method of claim 1, further comprising rehydrating the dried fibrin in an aqueous solution prior to use.

16. (canceled)

17. The method of claim 1, wherein the prosthetic device is an artificial bone implant.

18. The method of claim 1, wherein the prosthetic device comprises metallic material.

19. The method of claim 18, wherein the metallic material is titanium.

20. The method of claim 19, wherein the prosthetic device comprises titanium covered with titanium oxide.

21. A prosthetic device coated with dehydrothermal cross-linked fibrin prepared according to claim 1.

22. A method for treating a tissue defect or lesion in a mammalian subject comprising implanting into the tissue defect or lesion a prosthetic device coated with dehydrothermal cross-linked fibrin prepared according to claim 1.

23. The method according to claim 22, wherein the tissue defect or lesion is a bone lesion.

24. The method according to claim 23, wherein the bone lesion is a tooth lesion.

25. The method according to claim 22, wherein the tissue defect or lesion is a cartilage lesion.

26. (canceled)

27. A prosthetic device coated with dehydrothermal cross-linked fibrin for treating a tissue defect or lesion in a mammalian subject, the device coated with dehydrothermal cross-linked fibrin is prepared according to the method of claim 1.
